(19) United States
(12) Patent Application Publication
(10) Pub. No.: US 2010/0069939 A1
(43) Pub. Date: Mar. 18, 2010

(54) OPERATION SYSTEM

(75) Inventor: Sumihito KONISHI, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/210,796

(22) Filed: Sep. 15, 2008

(51) Int. Cl. A61B 17/32

(52) U.S. Cl. 606/169

(57) ABSTRACT

A surgery system includes: an ultrasonic output device for outputting to an ultrasonic transducer provided in a treatment instrument for performing surgery an ultrasonic drive signal to ultrasonically vibrate the ultrasonic transducer; a high-frequency output device for outputting a high-frequency signal for high-frequency ablation to the treatment instrument; and a connector section including a first connector and a second connector provided to the ultrasonic output device and the high-frequency output device, respectively, the connector section transmitting an output of the ultrasonic drive signal or the high-frequency signal from one of the devices to the other of the devices by connecting both of the connectors.

### BACKGROUND OF THE INVENTION

1. Field of the Invention

The present invention relates to a surgery system for performing a surgery using ultrasonic waves and high-frequency waves.

2. Description of Related Art

In recent years, there have been widely used ultrasonic (driving) output devices and high-frequency output devices (electrocautery devices) which perform a treatment on a living tissue and the like as an object to be treated using ultrasonic energy and high-frequency current, respectively.

FIG. 9 shows a configuration of a surgery system 81A in a first prior art example. The surgery system 81A includes a handpiece 82 used for a surgery and a high-frequency output device 85 and an ultrasonic output device 86 to which the high-frequency cable 83 and the ultrasonic cable 84, which are extended from a rear end of the handpiece 82, are connected, respectively. An ultrasonic transducer not shown is incorporated in the handpiece 82.

The high-frequency output device 85 and the ultrasonic output device 86 are connected through a communication cable 87.

One of the high-frequency output device 85 and the ultrasonic output device 86 can be selected to perform a treatment.

In addition, FIG. 10 shows a configuration of a surgery system 81B in a second prior art example similar to a disclosure in Japanese Patent Application Laid-Open Publication No. 6-343647. In the surgery system 81B, the handpiece 82 employs a cable 88 integrating the high-frequency cable 83 and the ultrasonic cable 84 in FIG. 10 as one cable, and a connector 89 of the cable 88 is connected only to an output connector 86a of the ultrasonic output device 86.

Furthermore, the high-frequency output device 85 and the ultrasonic output device 86 are connected by the communication cable 87 and further connected by a high-frequency cable 90 for transmitting a high-frequency signal. The high-frequency cable 90 connects between front panels of the high-frequency output device 85 and the ultrasonic output device 86 by connectors 91, 92.

Then, the high-frequency signal from the high-frequency output device 85 is supplied to the handpiece 82 through the high-frequency cable 90 and via the connector 89 connected to the ultrasonic output device 86.

In the surgery system 81B, a high-frequency signal and an ultrasonic drive signal for driving the ultrasonic transducer can be simultaneously outputted to the handpiece 82.

### SUMMARY OF THE INVENTION

A surgery system according to one aspect of the present invention includes: an ultrasonic output device for outputting to an ultrasonic transducer provided in a treatment instrument for performing surgery an ultrasonic drive signal to ultrasonically vibrate the ultrasonic transducer; a high-frequency output device for outputting a high-frequency signal for high-frequency ablation to the treatment instrument; and a connector section including a first connector provided to the ultrasonic output device and a second connector provided to the high-frequency output device, the connector section transmitting an output of the ultrasonic drive signal or the high-frequency signal from one of the devices to the other of the devices by connecting the first connector and the second connector.

A surgery system according to another aspect of the present invention includes: a treatment instrument incorporating an ultrasonic transducer that is ultrasonically vibrated by application of an ultrasonic drive signal, the treatment instrument being provided with a conductor portion for transmitting the ultrasonic vibration to a treatment portion at a distal end portion and also transmitting a high-frequency signal for high-frequency ablation to the treatment portion; an ultrasonic output device for outputting the ultrasonic drive signal; a high-frequency output device for outputting the high-frequency signal; a connector section to which a first connector provided to the ultrasonic output device and a second connector provided to the high-frequency output device are detachably connected, the connector section transmitting the ultrasonic drive signal or the high-frequency signal outputted from one of the ultrasonic output device and the high-frequency output device to the other of the devices; an output connector for outputting the ultrasonic drive signal and the high-frequency signal to a treatment instrument connector by connection with the treatment instrument connector provided to the treatment instrument; a connector connection detection section for detecting connection between the first connector and the second connector; and a control section for inhibiting outputs of the ultrasonic drive signal and the high-frequency signal to the treatment instrument connector when the connection between the first connector and the second connector is not detected.

### BRIEF DESCRIPTION OF THE DRAWINGS

FIG. 1 shows an appearance of a surgery device according to a first embodiment of the present invention.

FIG. 2 is a perspective view showing an appearance of an ultrasonic output device.

FIG. 3 is a cross-sectional view showing a situation where the high-frequency output device and the ultrasonic output device are connected by a docking connector.

FIG. 4A is a block diagram showing internal configurations of the high-frequency output device and the ultrasonic output device.

FIG. 4B is a block diagram showing internal configurations of the high-frequency output device and the ultrasonic output device.

FIG. 5 is a view showing an electrical configuration of a handpiece connector and an output connector.

FIG. 6 is a flowchart showing an operation example of the first embodiment.

FIG. 7 is a view showing configurations of main portions of the high-frequency output device and the ultrasonic output device according to a second embodiment of the present invention.

FIG. 8 is a flowchart showing a part of operations in the second embodiment.

FIG. 9 is a view showing a configuration of a surgery system according to a first prior art example.

FIG. 10 is a view showing a configuration of a surgery system according to a second prior art example.

### DETAILED DESCRIPTION OF PREFERRED EMBODIMENTS

Hereinafter, embodiments of the present invention will be described with reference to the drawings.

### First Embodiment

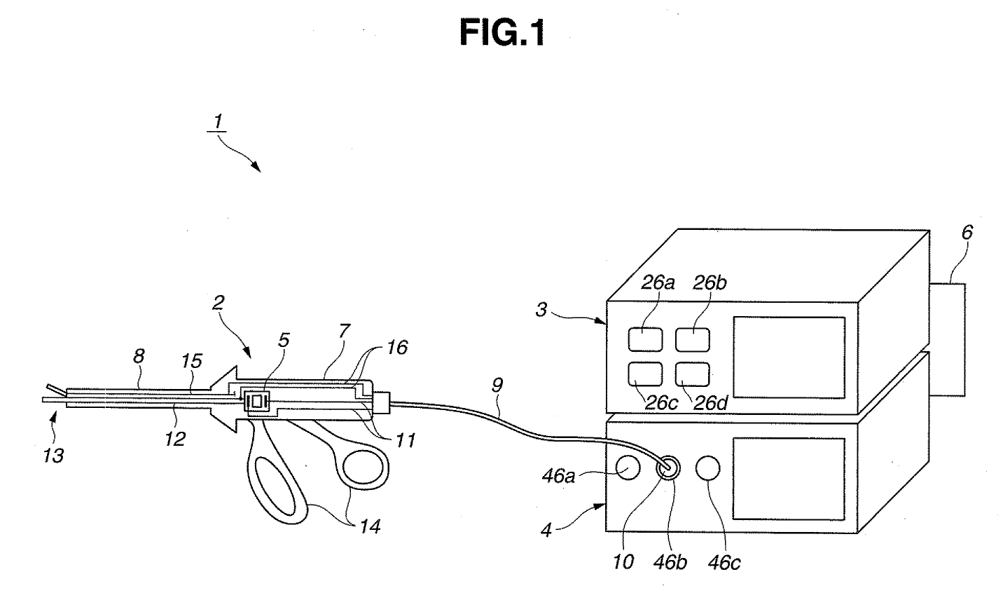

The first embodiment of the present invention will be described with reference to FIGS. 1 to 6.

As shown in FIG. 1, a surgery system 1 according to the first embodiment of the present invention includes: a handpiece 2 as a treatment instrument for performing a treatment on a living tissue as an object to be treated; a high-frequency output device 3 for outputting a high-frequency signal to the handpiece 2; and an ultrasonic output device 4 for outputting to an ultrasonic transducer 5 incorporated in the handpiece 2 an ultrasonic drive signal to ultrasonically vibrates the ultrasonic transducer.

In addition, the high-frequency output device 3 and the ultrasonic output device 4 are connected on their rear surface sides for example, through the communication cable 6.

The handpiece 2 includes a grasping portion 7 grasped by an operator for operation, and a sheath portion 8 extended forward from the grasping portion 7. A rear end of the grasping portion 7 is connected with a distal end of a cable 9, and a handpiece connector (abbreviated as HP connector) 10 at a rear end of the cable 9 is detachably connected to an output connector 46b of the ultrasonic output device 4.

The ultrasonic output device 4 is capable of supplying an ultrasonic drive signal to the ultrasonic transducer 5 in the grasping portion 7, through ultrasonic cables 11 in the cable 9. The supply of the ultrasonic drive signal ultrasonically vibrates the ultrasonic transducer 5. The ultrasonic vibration is transmitted to a distal end portion of the sheath portion 8 through a probe 12 in the sheath portion 8. Then the ultrasonic vibration energy generates frictional heat in a living tissue as an object to be treated, thereby enabling treatment such as coagulation, incision, and the like.

Note that, on a distal end side of the probe 12, a treatment portion 13 is formed by a distal end portion of the probe 12 and a movable piece which moves openably/closably with respect to the distal end portion.

The grasping portion 7 is provided with a finger-hooking portion 14 for performing an opening/closing operation. The operator performs the opening/closing operation with his or her fingers hooked on the finger-hooking portion 14, to pull a wire inserted through the sheath portion 8 and open/close the movable piece of the treatment portion 13, and thereby capable of grasping the living tissue as the object to be treated.

Furthermore, two high-frequency cables 16 for transmitting high-frequency signals are also inserted through the cable 9, one of distal ends of the high-frequency cables 16 is connected to a rear end of the probe 12 and the other is connected to a rear end side of the wire 15 (conducting to the movable piece). Note that the movable piece, the probe 12, and the wire 15 are formed by a conductive body of metal and the like for transmitting a high-frequency signal. The high-frequency cable 16 may be connected to the movable piece by a lead wire inserted through the sheath portion 8, instead of being connected to the rear end side of the wire 15. By applying a high-frequency current to the living tissue grasped by the treatment portion 13, a high-frequency ablation treatment can be performed.

Note that the handpiece 2 shown in FIG. 1 is a bipolar handpiece. In the case of a monopolar handpiece, one high-frequency cable 16 is connected to the rear end of the probe 12. In this case, a return path of the high-frequency current is formed by a return electrode not shown.

The rear ends of the ultrasonic cables 11 and the high-frequency cables 16 which are inserted through the cable 9 are connected to the output connector 46b of the ultrasonic output device 4 by the HP connector 10.

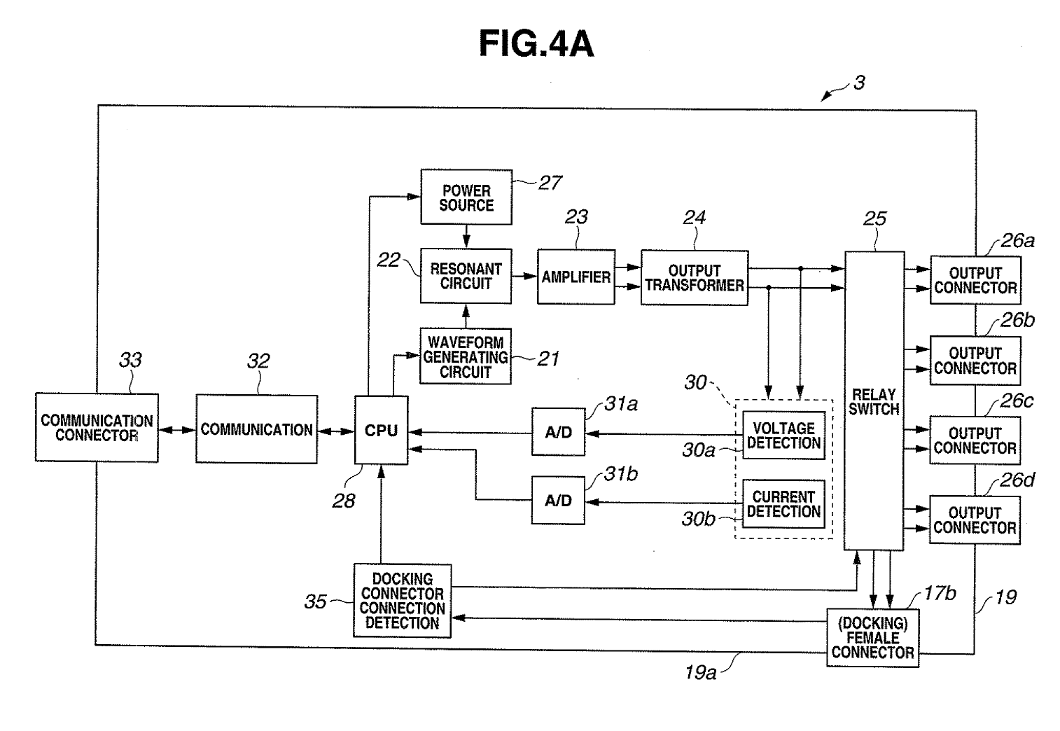

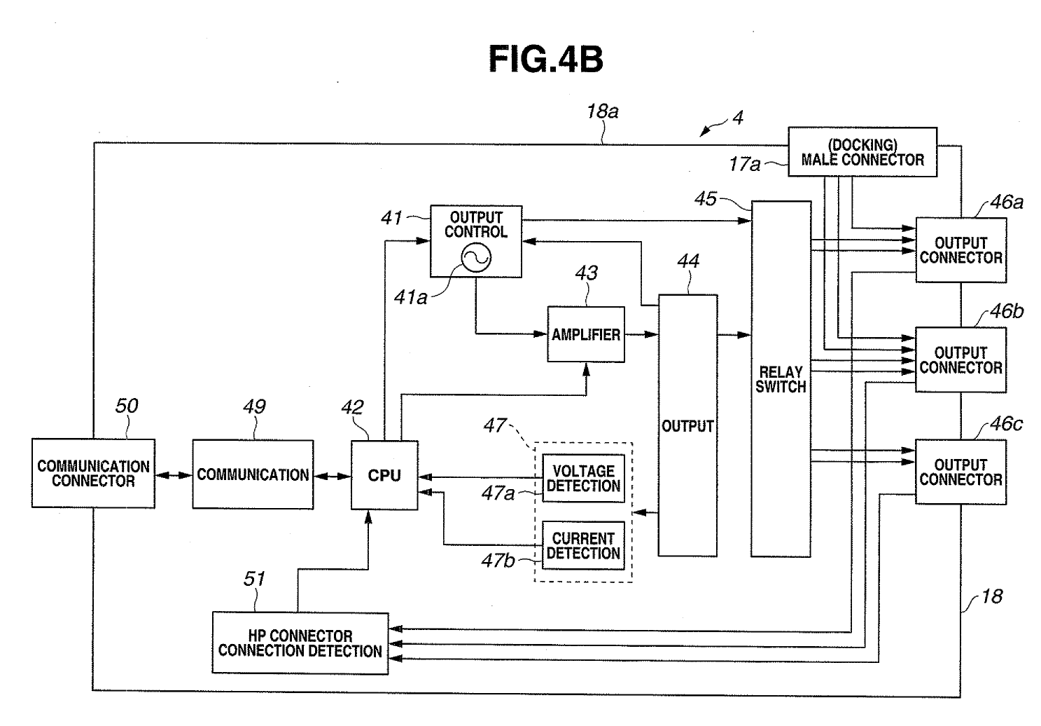

The ultrasonic cables 11 are connected to a relay switch circuit 45 shown in FIG. 4B through the output connector 46b. On the other hand, the high-frequency cables 16 are electrically connected to the high-frequency output device 3 (a relay switch circuit 25 inside thereof) through the output connector 46b and through a docking connector 17 shown in FIG. 3 as a connecting portion between the ultrasonic output device 4 and the high-frequency output device 3.

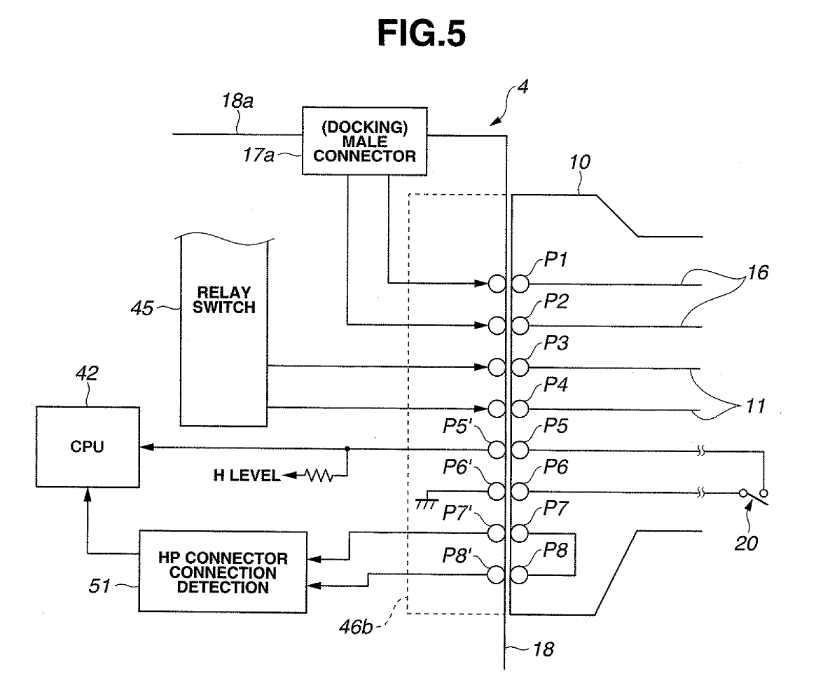

By turning on an output switch 20 (see FIG. 5) for performing an instruction operation of simultaneous outputs of ultrasonic waves and high frequency waves, ON-information of the output switch 20 is transmitted from (a CPU 42 of) the ultrasonic output device 4 to (a CPU 28 of) the high-frequency output device 3 through the communication cable 6, and a high-frequency signal and an ultrasonic drive signal are simultaneously outputted to the handpiece 2.

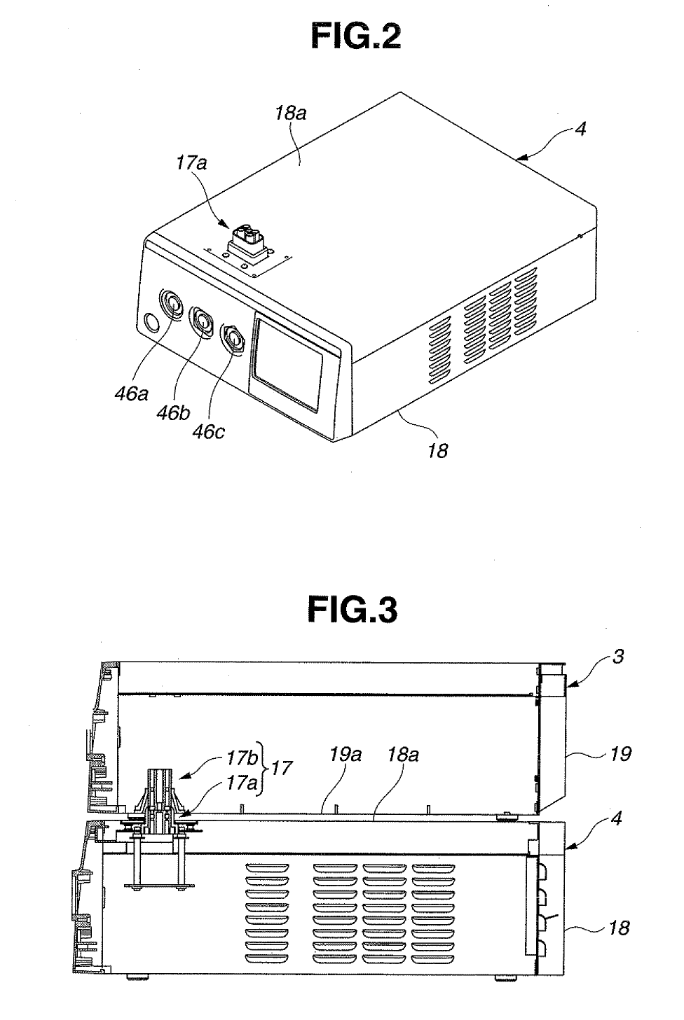

FIG. 2 shows a docking male connector (abbreviated as male connector) 17a configuring the docking connector 17 provided to a housing 18 as a storing case for the ultrasonic output device 4. For example, at a position near a front face (front panel) on a top plate 18a of the housing 18 is provided the male connector 17a having a connector pin projecting upward from the top plate 18a.

In addition, in a bottom plate 19a of a housing 19 of the high-frequency output device 3 is provided a docking female connector (abbreviated as female connector) 17b configuring the docking connector 17, as shown in FIG. 3. Then, as shown in FIG. 3, placing the housing 19 of the high-frequency output device 3 on the top plate 18a of the housing 18 of the ultrasonic output device 4 enables the docking of the male connector 17a and the female connector 17b which are provided at opposed positions on both plate surfaces, and thereby the devices can be set in a connection state.

In a prior art example shown in FIG. 10, the high-frequency signal outputted from the high-frequency output device 85 enters inside of the ultrasonic output device 86 via the high-frequency cable 90, and further from the inside of the ultrasonic output device 86, the high-frequency signal is transmitted to the handpiece 82 side through the cable 88 to which the connector 89 connected to the output connector 86a is connected.

In the present embodiment in contrast, the high-frequency signal outputted from the high-frequency output device 85 passes through from the female connector 17b provided in the bottom plate 19a of the housing 19 to the male connector 17a provided on the top plate 18a of the housing 18 of the ultrasonic output device 4 at the position opposed to the female connector 17b, and is transmitted to the handpiece 2 side, through the cable 9 to which the HP connector 10 connected to the output connector 46b is connected.

Thus, in the present embodiment, the high-frequency transmission path between the high-frequency output device 85 and the ultrasonic output device 86 in the prior art example is formed to be very short, by not using the high-frequency cable 90 which requires a long high-frequency transmission path. Therefore, the present embodiment is capable of reducing outside radiation of high-frequency signals that causes a noise source and a leak current.

In addition, the present embodiment unnecessitates wiring of the high-frequency cable on the front panel sides of the high-frequency output device 85 and the ultrasonic output device 86. Therefore, wiring of the high-frequency cable is unnecessary on the front panel sides which are frequently used by an operator, thereby enabling the operator to easily perform operations on the front panel sides. Furthermore, the present embodiment can prevent the high-frequency cable from interfering with the display on the front panel sides.

FIG. 4A shows a configuration of the high-frequency output device 3.

The high-frequency output device 3 incorporates a waveform generating circuit 21 for generating a sine wave and a burst wave, and a signal of the sine wave or burst wave outputted from the waveform generating circuit 21 is inputted to an amplifier 23 via a resonant circuit 22.

The signal amplified by the amplifier 23 is applied to a primary winding side of an output transformer 24, thereby generating a high-frequency (output) signal for ablation in a secondary winding side.

The secondary winding of the output transformer 24 is connected, for example, to four output connectors 26a, 26b, 26c and 26d, and the female connector 17b, through the relay switch circuit 25 for switching the high-frequency signals outputted from the secondary winding.

Note that the female connector 17b is provided in the bottom plate 19a of the housing 19 as described above. Furthermore, the resonant circuit 22 is supplied with a power source voltage from a voltage-variable power source circuit 27, and the waveform generating circuit 21 and the power source circuit 27 are controlled by the CPU 28 as a control section.

The CPU 28 controls the waveform generating circuit 21 and the power source circuit 27 according to output mode setting, output setting values and the like set by a setting section not shown.

Output signals from the secondary winding of the output transformer 24 are inputted to a voltage detection circuit 30a and a current detection circuit 30b which configure a detection section 30.

The voltage detection circuit 30a and the current detection circuit 30b detect (measure) voltage and current of the high-frequency signal outputted from the secondary winding of the output transformer 24. The detected voltage and current are converted by A/D converters 31a, 31b into digital voltage and current, to be inputted into the CPU 28.

The CPU 28 detects (calculates), from the inputted voltage and current, high-frequency power which is a product of the voltage and current. The CPU 28 controls the voltage supplied from the power source circuit 27 so that the detected high-frequency power value is equal to a setting value set by the setting section in advance.

In addition, the CPU 28 is connected to a communication connector 33 through a communication circuit 32 for performing communications. The communication connector 33 is connected to a communication connector 50 of the ultrasonic output device 4 side shown in FIG. 4B, through the communication cable 6.

The female connector 17b connected to the relay switch circuit 25 is detachably connected to the male connector 17a of the ultrasonic output device 4 side, as described above.

In addition, for example two connection detection connector pins in the female connector 17b are connected to a docking connector connection detection circuit 35. The docking connector connection detection circuit 35 constantly detects the connection between the male connector 17a and the female connector 17b using the connection detection connector pins.

In this case, the two connection detection connector pins are set so as to be connected, for example, to short-circuited two connector pins in the other connector side, i.e., the male connector 17a side.

Therefore, by detecting whether or not the two connection detection connector pins are in a conduction state, connection detection can be made as to whether or not the docking connector 17 is connected.

Then, the connection detection result by the docking connector connection detection circuit 35 is transmitted to the CPU 28. When the connection detection result by the docking connector connection detection circuit 35 indicates non-connection, the CPU 28 inhibits simultaneous execution of the ultrasonic (driving) output and the high-frequency output.

In other words, the CPU 28 permits the simultaneous execution of the ultrasonic output and the high-frequency output only when the connection of the docking connector 17 is detected.

In addition, when detecting the connection between the male connector 17a and the female connector 17b, the docking connector connection detection circuit 35 controls the switching of the relay switch circuit 25 so that the output signal from the output transformer 24 is outputted to the female connector 17b side. Note that, instead of the docking connector connection detection circuit 35, the CPU 28 may control the switching.

On the other hand, the ultrasonic output device 4 shown in FIG. 4B includes an output control circuit 41 incorporating an oscillation circuit 41a. The output control circuit 41 adjusts frequency and current of an oscillation signal oscillated by the oscillation circuit 41a and outputs the adjusted frequency and current to an amplifier 43, under control by a CPU 42 as a control section.

The signal amplified by the amplifier 43 is inputted to an output circuit 44 to be voltage-amplified by a transformer not shown in the output circuit 44, and outputted as an ultrasonic driving (output) signal from the secondary winding of the transformer The ultrasonic drive signal is connected to the three output connectors 46a, 46b and 46c, through the relay switch circuit 45 which switches and outputs the ultrasonic drive signal. Note that gain of the amplifier 43 is controlled by the CPU 42.

The two output connectors 46a, 46b are connected also to the male connector 17a. One of the two output connectors 46a and 46b, that is, the output connector 46b is connected with the bipolar handpiece 2, and the other, that is, the connector 46a is connected with the monopolar handpiece.

Note that the output connector 46c is not connected to the male connector 17a, but connected to a handpiece dedicated for ultrasonic output which outputs ultrasonic waves independently of the high-frequency output device 3.

The ultrasonic drive signal outputted from the output circuit 44 is inputted to a voltage detection circuit 47a and a current detection circuit 47b which configure a detection section 47, and voltage and current of the signal are detected (measured). The detected voltage and current are inputted to the CPU 42 through the AID converters in the voltage detection circuit 47a and the current detection circuit 47b, respectively.

In addition, there is provided a setting section, not shown, which sets power of the ultrasonic drive signal to be supplied to the ultrasonic transducer 5 of the handpiece 2, and information on the setting is inputted to the CPU 42.

The CPU 42 performs constant current control through the output control circuit 41 based on the voltage and current detected through the detection section 47 such that the power set by the setting section is outputted from the output circuit 44.

For this end, the CPU 42 temporarily retains in a memory in the output control circuit 41 the control information on the output value in outputting power from the output circuit 44, and based on the voltage and current detected thereafter, the CPU 42 performs control to correct immediately preceding control information through the output control circuit 41.

Furthermore, the CPU 42 is connected to the communication connector 50 through a communication circuit 49 for performing communications. The communication connector 50 is connected to the communication connector 33 on the high-frequency output device 3 side shown in FIG. 4A, through the communication cable 6. The CPU 42 and the CPU 28 can communicate bidirectionally through the communication cable 6.

In addition, connector connection detection pins in the three output connectors 46a, 46b and 46c are connected to an HP connector connection detection circuit 51. The HP connector connection detection circuit 51 detects connection/non-connection of the HP connector 10.

Note that, as described above, the bipolar handpiece 2 is connected to the output connector 46b and the monopolar handpiece is connected to the output connector 46a. The HP connector connection detection circuit 51 sends information on detection result to the CPU 42.

Based on the information on the detection result, the CPU 42 controls switching of the relay switch circuit 45 through the output control circuit 41 such that the output signal (that is, ultrasonic drive signal) from the output circuit 44 is supplied to the output connector to which the handpiece is connected. Note that the CPU 42 may control the switching of the relay switch circuit 45.

FIG. 5 shows a configuration of the HP connector 10. Connector pins P1, P2 are connected to the male connector 17a via the output connector 46b. Connector pins P3, P4 are connected to the relay switch circuit 45 via the output connector 46b.

In addition, connector pins P5, P6 connected to the output switch 20 provided to the handpiece 2 are connected to connector pins P5′, P6′ on the output connector 46b side. In the example of FIG. 5, the connector pin P6′ is grounded and the connector pin P5′ is connected to the CPU 42. In this case, the connector pin P5′ is pulled up to an H level by a resistor, for example. When the output switch 20 is turned on, the level of the connector pin P5′ becomes an L level from the H level, and the CPU 42 detects that the output switch 20 was turned on. The CPU 42 sends the signal indicating that the output switch 20 was turned on to the CPU 28 in the high-frequency output device 3 through the communication cable 6 to cause the CPU 28 to output a high-frequency signal, and outputs an ultrasonic drive signal.

In addition, connector pins P7, P8 are connection detection pins and are short-circuited Connector pins P7′, P8′ on the output connector 46b side, to which both of the connector pins P7, P8 are to be connected, are connected to the HP connector connection detection circuit 51.

The HP connector connection detection circuit 51 makes connection detection as to whether or not the HP connector 10 is connected to the output connector 46b, based on whether or not the signal state indicates that the connector pins P7′ and P8′ are short-circuited or open therebetween.

Note that the detection as to whether or not the HP connector 10 is connected to the output connector 46b may be performed by utilizing variation of signal levels due to the connection/non-connection of the HP connector 10, same as in the ON/OFF detection of the output switch 20, instead of the signal detection by resistance values corresponding to the short-circuited/open state of the connector pins. Also the above-described docking connector connection detection circuit 35 can detect the connection between the male connector 17a and the female connector 17b with the same configuration.

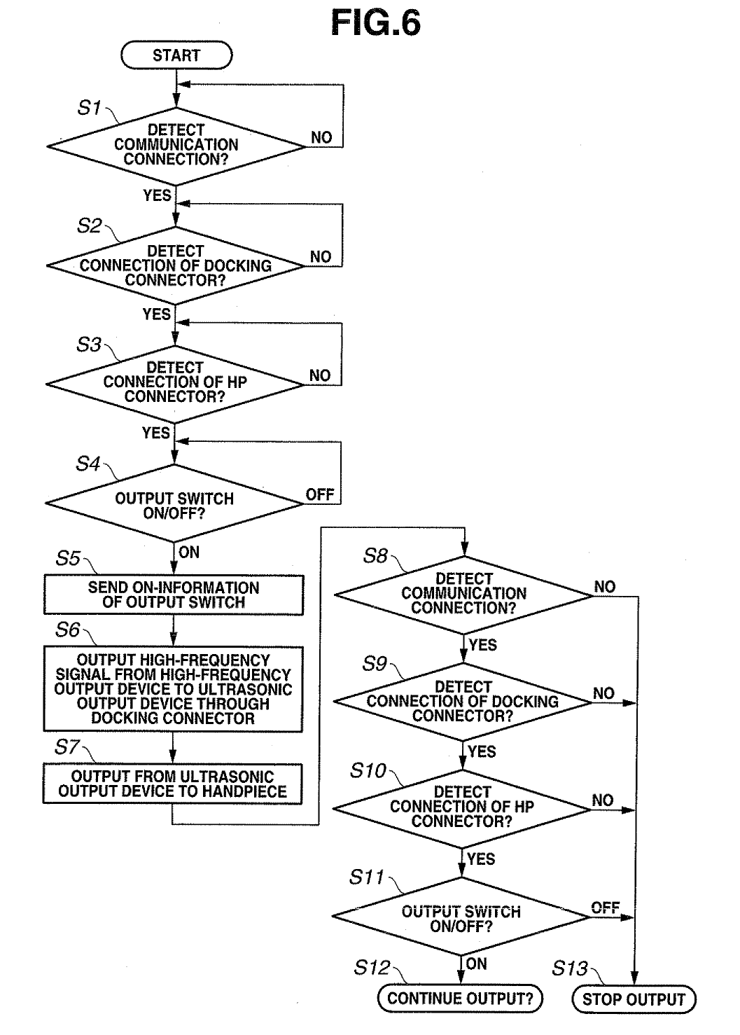

An operation of the surgery system 1 having such a configuration will be described with reference to FIG. 6.

As shown in FIG. 1, the operator first connects the connector 10 of the cable 9 connected to the handpiece 2 to the ultrasonic output device 4, and also connects the high-frequency output device 3 and the ultrasonic output device 4 by the communication cable 6.

Then, the operator turns on the power source switches of the high-frequency output device 3 and the ultrasonic output device 4. This allows the high-frequency output device 3 and the ultrasonic output device 4 to be in an operation state, and thereby the communication connection detection of step S1 shown in FIG. 5 is started.

One of the CPU 28 in the high-frequency output device 3 and the CPU 42 in the ultrasonic output device 4 sends a signal for connection detection to the other of the CPUs through the communication cable 6 and receives notification of reception of the signal from the other of the CPUs, and thereby detecting whether or not the devices are in a communicable connection state, to wait until the devices become a connection state. Note that the connection may be detected depending on whether or not the communication is possible.

When the high-frequency output device 3 and the ultrasonic output device 4 are connected by the communication cable 6 as shown in FIG. 1, the connection (state) between the devices is detected.

When the connection is detected, in the next step S2, the docking connector connection detection circuit 35 provided in the high-frequency output device 3 detects whether or not the docking connector 17 is in a connection state and waits until the docking connector becomes a connection state.

When the docking connector 17 is set in the connection state as shown in FIG. 3, the docking connector connection detection circuit 35 detects the connection state.

When the docking connector 17 becomes a connection state, in the next step S3, the HP connector connection detection circuit 51 provided in the ultrasonic output device 4 detects whether or not the HP connector 10 of the handpiece 2 is connected to the output connector 46b, and waits until the HP connector 10 becomes a connection state. As shown in FIG. 1, when the HP connector 10 of the handpiece 2 is connected to the output connector 46b, the connection state is detected.

Then, the high-frequency output device 3 and the ultrasonic output device 4 become ready to output, and wait until the output switch 20 is turned on in the next step S4.

When the output switch 20 is turned on by the operator, in the next step S5, the CPU 42 in the ultrasonic output device 4 sends the ON-information of the output switch 20 to the CPU 28 in the high-frequency output device 3 via the communication cable 6.

When receiving the ON-information, the CPU 28 in the high-frequency output device 3 immediately outputs a high-frequency signal in step S6. That is, the high-frequency signal is transmitted (outputted) from the high-frequency output device 3 to the ultrasonic output device 4 through the docking connector 17. The transmitted high-frequency signal is further outputted to the handpiece 2 via the output connector 46b and the HP connector 10.

Furthermore, simultaneously in the step S7, the ultrasonic output device 4 outputs an ultrasonic drive signal to the handpiece 2.

The operator operates the handpiece 2 and grasps a living tissue as an object to be treated with the treatment portion 13, to perform treatment such as resection by high-frequency energy and ultrasonic vibration energy.

In the next step S8, the CPU 28 and the CPU 42 detect communication connection same as in the step S1. When the communication connection cannot be detected, the CPUs stop (or inhibit) the outputs of high frequency waves and ultrasonic waves as shown in step S13 (same as in the case where the output switch 20 is turned off).

When the connection has been detected, the connection of the docking connector 17 is detected in the next step S9 same as in the step S2.

When the connection cannot be detected, the outputs of high frequency waves and ultrasonic waves are stopped. When the connection is detected, the connection of the handpiece is detected in the next step S10 same as in the step S3.

When the connection cannot be detected, the outputs of high frequency waves and ultrasonic waves are stopped. When the connection has been detected, the determination as to whether the output switch is turned on or off is made in the next step S11 same as in the step S4.

When the output switch 20 is turned off, the outputs of high frequency waves and ultrasonic waves are stopped. When the output switch is turned on, the outputs of high frequency waves and ultrasonic waves are continued as shown in step S12.

According to the present embodiment thus operates, the transmission path of the high-frequency signal can be made sufficiently short, thereby enabling noise reduction and leak current suppression. As a result, excellent electric characteristics can be obtained.

In addition, a user such as the operator and the like has only to connect the HP connector 10 to the output connector of the ultrasonic output device 4 at one position, thereby reducing the connection labor Therefore, the present embodiment can ensure excellent operability.

In addition, since the present embodiment requires only one piece of the communication cable 6 for connecting the high-frequency output device 3 and the ultrasonic output device 4, the connection labor is reduced. In this case, the communication cable 6 does not interfere with the display.

Furthermore, since the communication cable 6 does not occupy the existing connectors of the high-frequency output device 3 side in the present embodiment, there is no limitation placed on the number of devices connectable to the high-frequency output device 3.

Moreover, since the present embodiment has a structure in which the docking connector connection is made at a position where the operator and the like cannot touch, the devices are not easily detached once they are connected. Therefore, the present embodiment can reduce or resolve disconnection of cables in use and breaking of wires caused by repeated cable use, which can often occur in connections using a cable.

### Second Embodiment

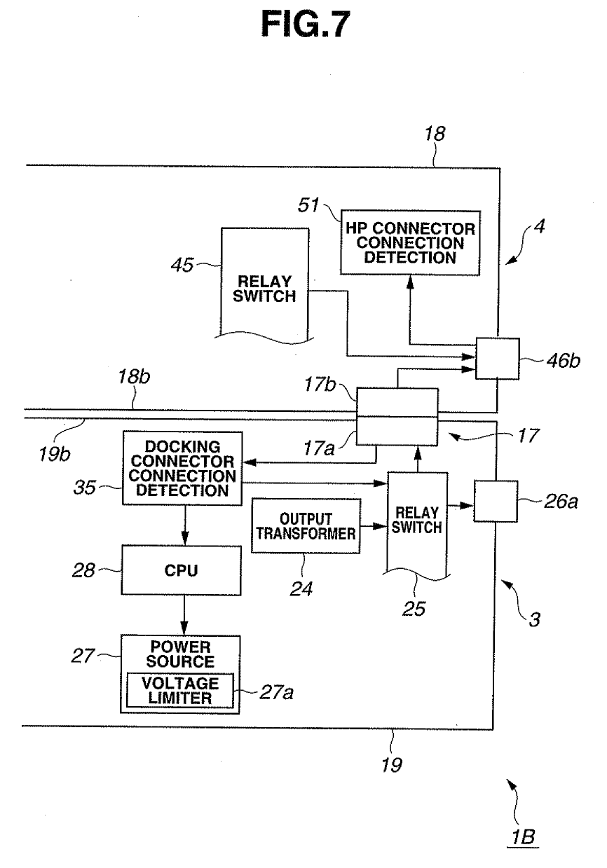

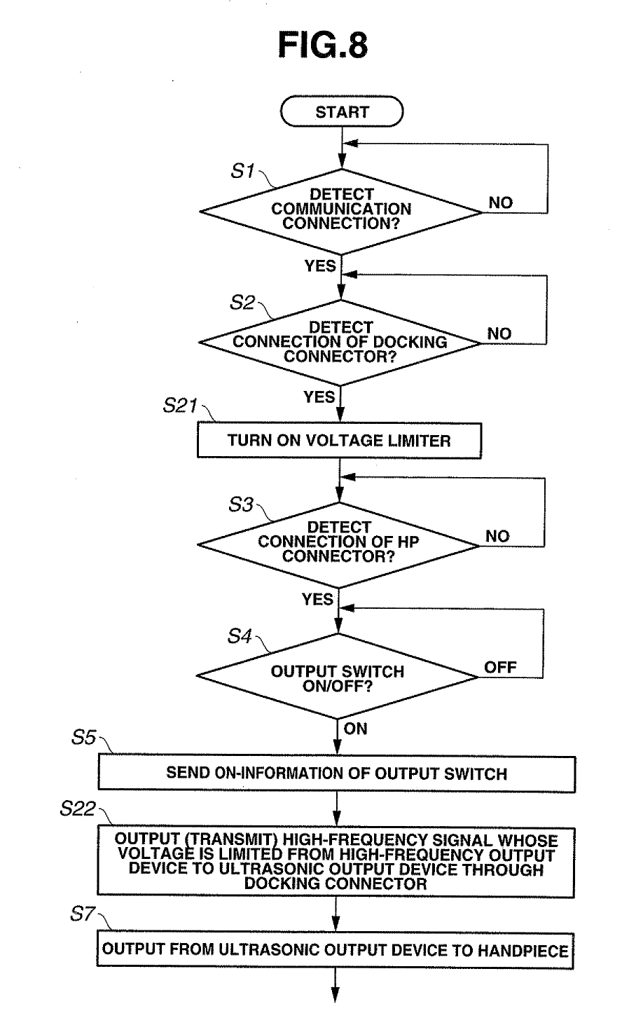

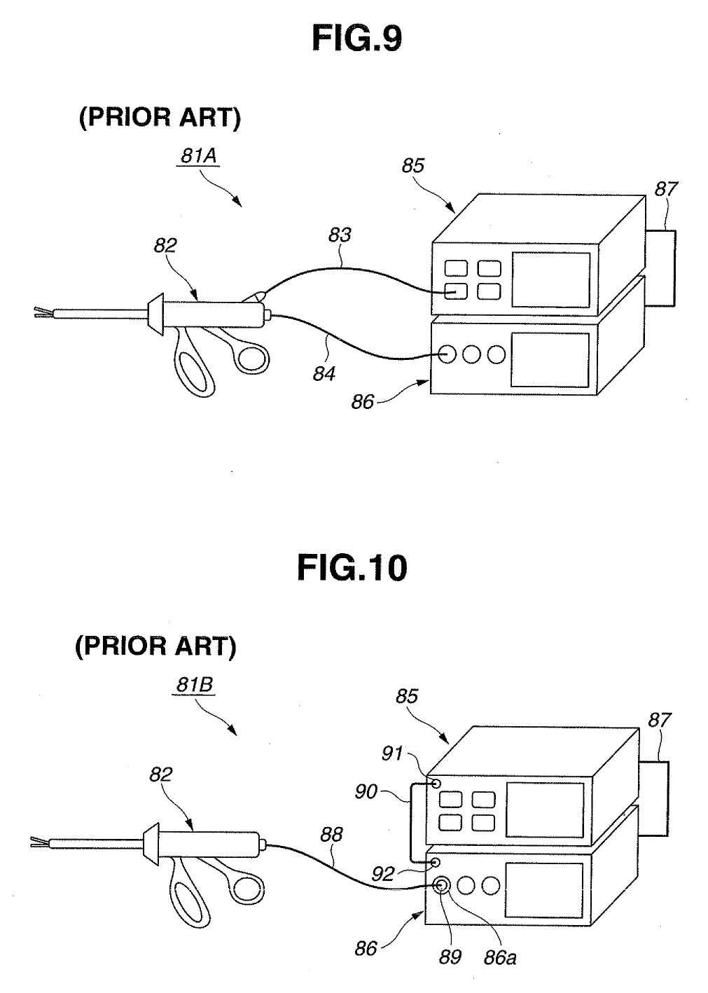

Next, the second embodiment of the present invention will be described with reference to FIGS. 7 and 8. In the first embodiment, the docking connector 17 is configured of the male connector 17a provided on the top plate 18a of the housing 18 of the ultrasonic output device 4 and the female connector 17b provided in the bottom plate 19a of the housing 19 of the high-frequency output device 3.

In contrast, a surgery system 1B according to the present embodiment has a configuration in which the relationship between the both devices in the first embodiment is reversed.

FIG. 7 shows a schematic configuration of the main parts of the high-frequency output device 3 and the ultrasonic output device 4 according to the present embodiment.

In the present embodiment, the docking connector 17 is configured of the male connector 17a provided on the top plate 19b of the housing 19 of the high-frequency output device 3 and the female connector 17b provided in the bottom plate 18b of the housing 18 of the ultrasonic output device 4.

Note that the connection structure of the male connector 17a and the female connector 17b may be reversed in the devices. In addition, for example the output connector 46b is provided at a position in the vicinity of the bottom surface of the front face of the housing 18 in the present embodiment. That is, the output connector 46b is provided at a position spaced a short distance from the docking connector 17. The transmission path of high-frequency signal is made as short as possible.

Note that also the output connector 46a to which the monopolar handpiece is connected is similarly provided at a position in the vicinity of the bottom surface of the front face of the housing 18.

Note that the output connector 46a is located at an upper or lower vertical position with respect to the paper surface in FIG. 7.

Furthermore, similarly as in the first embodiment, when detecting the connection of the docking connector 17, the docking connector connection detection circuit 35 transmits the information on the detection to the CPU 28 and switches the relay switch circuit 25 so that an output signal from the output transformer 24 is outputted to the docking connector 17 side. Then, the docking connector connection detection circuit 35 causes a high-frequency signal to be outputted to the output connector side of the ultrasonic output device 4.

In the present embodiment, the CPU 28 further performs a control to place a limit on the voltage outputted from the power source circuit 27 to the resonant circuit 22 such that the voltage (amplitude) of the high-frequency signal outputted from the output transformer 24 is equal to or less than a predetermined voltage value. The power source circuit 27 has a function of a voltage limiter 27a which limits the power source voltage to be outputted to an instructed voltage based on a voltage-limiting control signal from the CPU 28.

Other configurations are the same as those in the first embodiment.

Next, operations of the present embodiment will be described with reference to FIG. 8. The operations according to the present embodiment are similar to those shown in the flowchart in FIG. 6. Therefore, the operations will be described with reference to FIG. 6.

Steps S1, S2 in FIG. 8 are the same as the steps S1, S2 in FIG. 6. When detecting the connection of the docking connector in the step S2, the CPU 28 in the high-frequency output device 3 sends a control signal to place a limit on the power source voltage of the power source circuit 27 as shown in step S21. The power source circuit 27 then turns on the function of the voltage limiter. Thereafter, the same processings shown in the steps S3, S4 and S5 in FIG. 6 are performed.

When the output switch 20 is turned on in the step S4, the CPU 42 in the ultrasonic output device 4 sends ON-information of the switch 20 to the CPU 28 in the high-frequency output device 3 in the step S5.

Then, as shown in step S22 in FIG. 8, the high-frequency output device 3 outputs a high-frequency signal to the ultrasonic output device 4 through the docking connector 17.

In this case, since the voltage limiter is turned on, the high-frequency signal outputted from the high-frequency output device 3 to the ultrasonic output device 4 through the docking connector 17 has a voltage (amplitude) whose value is limited equal to or less than a predetermined value by the voltage limiter

The step S7, which is the next step of the step S22, and the steps thereafter are the same as those shown in FIG. 6. Therefore, the descriptions thereof will be omitted.

The present embodiment has the same merits as those in the first embodiment. In addition, since the output connector to which the HP connector 10 of the handpiece 2 is connected is provided in the vicinity of the docking connector 17, the noise to be radiated peripherally as well as the leak current can be reduced.

In addition, the present embodiment has a configuration in which the high-frequency signal is outputted by placing a limit on the voltage thereof.

When the output of the electrocautery is outputted from the ultrasonic device, reference voltage in a withstand voltage test is not the voltage of the ultrasonic waves but the voltage of the electrocautery. Then, the device has to pass the withstand voltage test according to the voltage of the electrocautery. As a result, hurdles for the design of the internal structure (electric circuits) of the ultrasonic device side become higher and also the cost of the device will be increased. In addition, in a noise resistance test for example, test conditions sometimes differ between the electrocautery and the ultrasonic device, in order to meet separate standards.

Furthermore, since the high-frequency output device generally uses an output voltage higher than that in the ultrasonic output device, the secondary circuit and the external packaging are insulated in the high-frequency output device in order to ensure user safety. Therefore, the configuration of the high-frequency output device is different from that of the ultrasonic output device. Accordingly, if the voltage of the high-frequency output device is applied as-is to the ultrasonic output device, the safety of the ultrasonic output device cannot be ensured. In order to solve this problem, the voltage limiter is used to place a limit on the voltage outputted to the docking connector, thereby allowing the safety to be ensured.

It is thus important to place a limit on the voltage outputted to the docking connector by using the limiter when the output of the electrocautery is outputted from the ultrasonic device.

Note that the configuration in which the ultrasonic drive signal and the high-frequency signal are outputted from the ultrasonic output device 4 side to the handpiece 2 is described in the above-described embodiment. However, the high-frequency signal and the ultrasonic drive signal may be outputted from the high-frequency output device 3 side to the handpiece 2.

That is, the output connectors 46a, 46b which output the ultrasonic drive signal and the high-frequency signal to the handpiece 2 may be provided in the high-frequency output device 3.

In this case, the docking connector 17 transmits (outputs) the ultrasonic drive signal from the ultrasonic output device 4 side to the high-frequency output device 3 side.

Accordingly, the present invention is applicable to the case where the ultrasonic drive signal and the high-frequency signal are transmitted (outputted) from one of the ultrasonic output device 4 and the high-frequency output device 3 to the other by the docking connector 17 as a connector section by which the both devices are connected.

In addition, in the above-described embodiment, the high-frequency output device 3 and the ultrasonic output device 4 are connected by the communication cable 6 at a position different from the position of the docking connector 17, for example, on the rear surface side of both of the housings. However, the end portion of the communication cable 6 may be connected to the connector pins of the docking connector 17. That is, communications may be performed between the ultrasonic output device 4 and the high-frequency output device 3 by using the docking connector 17.

Furthermore, the docking connector connection detection circuit 35 and the HP connector connection detection circuit 51 are not limited to those described in FIG. 5, and may be an optical switch or mechanical switch which utilizes a variation in light amount and the like between light-emitting devices and light-receiving elements caused by connection/non-connection (detachment) between detachably connected connectors. In addition, a current sensor may be provided on an output transmission line of the high-frequency signal so that the current sensor monitors to detect the connection.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. A surgery system comprising:
an ultrasonic output device for outputting to an ultrasonic transducer provided in a treatment instrument for performing surgery an ultrasonic drive signal to ultrasonically vibrate the ultrasonic transducer;
a high-frequency output device for outputting a high-frequency signal for high-frequency ablation to the treatment instrument; and
a connector section including a first connector provided to the ultrasonic output device and a second connector provided to the high-frequency output device, the connector section transmitting an output of the ultrasonic drive signal or the high-frequency signal from one of the devices to the other of the devices by connecting the first connector and the second connector.

2. The surgery system according to claim 1, further comprising
a connector connection detection section for detecting connection between the first connector of the ultrasonic output device and the second connector of the high-frequency output device.

3. The surgery system according to claim 2, further comprising
a control section for permitting the output of the ultrasonic drive signal or the high-frequency signal from one of the devices to the other of the devices when the connection between the first connector and the second connector is detected by the connector connection detection section, and inhibiting the output when the connection is not detected.

4. The surgery system according to claim 1, wherein
the first connector is provided to a first housing for storing the ultrasonic output device, the second connector is provided on a surface opposing the first housing of a second housing for storing the high-frequency output device, and one of the first connector and the second connector is configured of a male connector and the other is configured of a female connector.

5. The surgery system according to claim 4, wherein the male connector is provided on a top plate of one of the first housing and the second housing, and the female connector is provided in a bottom surface of the other of the first housing and the second housing.

6. The surgery system according to claim 2, wherein the connector connection detection section is provided to the first connector or the second connector configuring the connector section and detects the connection between the first connector and the second connector by utilizing a signal generated when the first connector and the second connector are connected.

7. The surgery system according to claim 2, wherein the connector connection detection section detects the connection between the first connector and the second connector during an operation of outputting the ultrasonic drive signal by the ultrasonic output device and during an operation of outputting the high-frequency signal by the high-frequency output device.

8. The surgery system according to claim 1, wherein the connector section transmits the high-frequency signal outputted from the high-frequency output device to the ultrasonic output device through the second connector and the first connector connected to the second connector.

9. The surgery system according to claim 8, further comprising:
a connector connection detection section for detecting the connection between the first connector of the ultrasonic output device and the second connector of the high-frequency output device; and
a voltage limiting section for limiting an output voltage value of the high-frequency signal transmitted from the high-frequency output device to the ultrasonic output device when the connector connection detection section detects the connection between the first connector and the second connector.

10. The surgery system according to claim 1, wherein the other of the devices includes an output connector to which a treatment instrument connector provided to the treatment instrument is connected, and the ultrasonic drive signal and the high-frequency signal are outputted from the output connector to the treatment instrument connector.

11. The surgery system according to claim 10, wherein the other of the devices includes a treatment instrument connector connection detection section for detecting whether or not the treatment instrument connector is connected to the output connector.

12. The surgery system according to claim 10, wherein outputs of the ultrasonic drive signal and the high-frequency signal are inhibited when the treatment instrument connector is not connected to the output connector.

13. The surgery system according to claim 8, wherein the ultrasonic output device includes an output connector to which a treatment instrument connector provided to the treatment instrument is connected, and the ultrasonic drive signal and the high-frequency signal are outputted from the output connector to the treatment instrument connector.

14. The surgery system according to claim 8, further comprising a connector connection detection section for detecting the connection between the first connector of the ultrasonic output device and the second connector of the high-frequency output device.

15. The surgery system according to claim 1, wherein the connector section incorporates a communication connection pin to perform communications between the ultrasonic output device and the high-frequency output device.

16. The surgery system according to claim 2, wherein
the one of the devices includes a switch circuit for selectively outputting the ultrasonic drive signal or the high-frequency signal to one of the connector section side and an output connector side provided to the one of the devices, and
the switch circuit is switched to allow the ultrasonic drive signal or the high-frequency signal to be outputted to the connector section side when the connection between the first connector and the second connector is detected by the connector connection detection section.

17. A surgery system comprising:
a treatment instrument incorporating an ultrasonic transducer that is ultrasonically vibrated by application of an ultrasonic drive signal, the treatment instrument being provided with a conductor portion for transmitting the ultrasonic vibration to a treatment portion at a distal end portion and also transmitting a high-frequency signal for high-frequency ablation to the treatment portion;
an ultrasonic output device for outputting the ultrasonic drive signal;
a high-frequency output device for outputting the high-frequency signal;
a connector section to which a first connector provided to the ultrasonic output device and a second connector provided to the high-frequency output device are detachably connected, the connector section transmitting the ultrasonic drive signal or the high-frequency signal outputted from one of the ultrasonic output device and the high-frequency output device to the other of the devices;
an output connector for outputting the ultrasonic drive signal and the high-frequency signal to a treatment instrument connector by connection with the treatment instrument connector provided to the treatment instrument;
a connector connection detection section for detecting connection between the first connector and the second connector; and
a control section for inhibiting outputs of the ultrasonic drive signal and the high-frequency signal to the treatment instrument connector when the connection between the first connector and the second connector is not detected.

18. The surgery system according to claim 17, further comprising
a treatment instrument connector connection detection section for detecting whether or not the treatment instrument connector is connected to the output connector, wherein the outputs of the ultrasonic drive signal and the high-frequency signal to the treatment instrument connector are inhibited when the treatment instrument connector is not connected to the output connector.

19. The surgery system according to claim 17, wherein when the one of the devices is the high-frequency output device, the high-frequency output device limits an output voltage value of the high-frequency signal to be equal to or less than a predetermined value when transmitting the high-frequency signal to the ultrasonic output device as the other of the devices through the connector section.

20. The surgery system according to claim 17, further comprising
a communication section for performing communications between the ultrasonic output device and the high-frequency output device, wherein
the control section inhibits the outputs of the ultrasonic drive signal and the high-frequency signal to the treatment instrument connector when communications are impossible between the ultrasonic output device and the high-frequency output device.
